(19) United States
(12) Patent Application Publication
(10) Pub. No.: US 2012/0040923 A1
(43) Pub. Date: Feb. 16, 2012

(54) STABLE S-ADENOSYL-L-METHIONINE

(76) Inventor: Rolland F. Hebert, Seattle, WA (US)

(21) Appl. No.: 13/283,412

(22) Filed: Oct. 27, 2011

Related U.S. Application Data
(62) Division of application No. 12/234,617, filed on Sep. 20, 2008.
Application No. 12/234,617 is a continuation-in-part of application No. 11/136,271, filed on May 24, 2005, now abandoned.

(51) Int. Cl. A61K 31/7076; A61P 19/02; A61P 37/00; A61P 9/00; C12P 19/40; A61P 35/04; A61P 25/00; A61P 25/24; A61P 1/16; A61P 27/02; A61P 25/28; A61P 35/00

(52) U.S. Cl. 514/46; 435/88

(57) ABSTRACT

Stable compositions of defined non-racemic diastereomeric ratios of S-adenosyl-L-methionine, methods for their synthesis and methods for their uses are described. The compositions according to the invention are very stable and are valuable for use as active constituents in pharmaceutical compositions.

### CROSS-REFERENCES TO RELATED APPLICATIONS

This application is a divisional of U.S. patent application Ser. No. 12/234,617, filed Sep. 20, 2008 (pending); which is a continuation-in-part of U.S. patent application Ser. No. 11/136,271 filed May 24, 2005 (now abandoned). These applications are incorporated herein by reference in their entireties.

### FIELD OF THE INVENTION

The present invention relates to synthetic methods as well as to uses of non-racemic mixtures of S-adenosyl-L-methionine diastereomers and their pharmaceutically acceptable salts, and more particularly, to the use of non-racemic mixtures of (S,S)S-adenosyl-L-methionine and (R,S)S-adenosyl-L-methionine and their pharmaceutically acceptable salts in the treatment of conditions associated with low blood and tissue levels of S-adenosyl-L-methionine, and low cellular, DNA or RNA methylation levels.

### BACKGROUND OF THE INVENTION

S-adenosyl-L-methionine is a naturally occurring substance that is present in all living organisms and has a number of very important biological functions. S-adenosyl-L-methionine exists in two important diastereomeric forms as (S,S)S-adenosyl-L-methionine and (R,S)S-adenosyl-L-methionine. Among these functions are the following: methyl group donor in transmethylation reactions (it is the sole methyl group donor in such reactions—including methylation of DNA and RNA, proteins, hormones, catechol and indoleamines and phosphatidylethanolamine to phosphatidylcholine); it is a substrate of an enzyme lyase that converts S-adenosyl-L-methionine to the molecule methylthioadenosine and homoserine; it is an aminobutyric chain donor to tRNA; it is an aminoacidic chain donor in the biosynthesis of biotin; S-adenosyl-L-methionine, after decarboxylation, is the donor of aminopropyl groups for the biosynthesis of neuroregulatory polyamines spermidine and spermine. (Zappia et al (1979) Biomedical and Pharmacological roles of Adenosylmethionine and the Central Nervous System, page 1, Pergamon Press. NY.)

However, in mammals, the (R,S)S-adenosyl-L-methionine diastereomer is found in quite low concentrations, roughly at 3% relative to the (S,S)S-adenosyl-L-methionine diastereomer. (Hoffman, 1986 Biochemistry, vol. 25, no 15, pp 4444-4449.)

The (R,S)S-adenosyl-L-methionine diastereomer has been reported to have an opposite activity to that of the (S,S))S-adenosyl-L-methionine diastereomer. (Borchardt and Wu, Journal of Medicinal Chemistry, 1976, Vol. 19, No. 9, pp 1099 to 1103.) Thus, it is of particular interest to develop compositions that have one or the other of the diastereomers in higher concentrations relative to the other when clinical conditions so dictate.

### Clinical Uses of S-Adenosyl-L-Methionine:

S-adenosyl-L-methionine has been used clinically for more than twenty years in the treatment of liver disease (Friedel H, Goa, K. L., and Benfield P., (1989) S-adenosyl-L-methionine: a review of its pharmacological properties and therapeutic potential in liver dysfunction and affective disorders in relation to its physiological role in cell metabolism. Drugs. 38, 389-416), arthritis (Di Padova C, (1987) S-adenosyl-L-methionine in the treatment of osteoarthritis: review of the clinical studies. Am J. Med. 83, (Suppl. 5), 6-65), and depression (Kagan, B, Sultzer D. L., Rosenlicht N and Gerner R. (1990) Oral S-adenosylmethionine in depression: a randomized, double-blind, placebo-controlled trial. Am. J. Psychiatry 147, 591-595.)

Alzheimer's patients have reduced cerebral spinal fluid levels of S-adenosyl-L-methionine (Bottiglieri et al, (1990) Cerebrospinal fluid S-adenosyl-L-methionine in depression and dementia: effects of treatment with parenteral and oral S-adenosyl-L-methionine. J. Neurol. Neurosurg. Psychiatry 53, 1096-1098.)

Brain 5-adenosylmethionine levels are severely decreased in Alzheimer's disease, Journal of Neurochemistry, 67, 1328-1331. Patients with Parkinson's disease have also been shown to have significantly decreased blood levels of S-adenosyl-L-methionine. (Cheng et al, (1997) Levels of L-methionine S-adenosyltransferase activity in erythrocytes and concentrations of S-adenosylmethionine and S-adenosylhomocysteine in whole blood of patients with Parkinson's disease. Experimental Neurology 145, 580-585.) Oral S-adenosyl-L-methionine administration to patients with and without liver disease has resulted in increases in liver glutathione levels. (Vendemiale G et al, Effect of oral S-adenosyl-L-methionine on hepatic glutathione in patients with liver disease. Scand J Gastroenterol 1989; 24: 407-15. Oral administration of S-adenosyl-L-methionine to patients suffering from intrahepatic cholestasis had improvements in both the pruritus as well as the biochemical markers of cholestasis. (Giudici et al, The use of admetionine (S-adenosyl-L-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral S-adenosyl-L-methionine administration to patients suffering from primary fibromyalgia resulted in significant improvement after a short term trial. (Tavoni et al, Evaluation of S-adenosylmethionine in Primary Fibromyalgia. The American Journal of Medicine, Vol 83 (suppl 5A), pp 107-110, 1987.) Lee Hong Kyu disclosed in a patent application WO02092105 (Nov. 21, 2002) that S-adenosyl-L-methionine could be used to treat diabetes and insulin resistance. A recently published evidence report entitled “S-adenosyl-L-methionine for the treatment of depression, osteoarthritis and liver disease” provides both safety and clinical efficacy data for this important biomolecule. (Evidence Report number 64, US Department of Health and Human Services, Public Health Service, Agency for Healthcare Research and Quality. October 2002.

S-adenosyl-L-methionine is clinically useful in many apparently unrelated areas because of its important function in basic metabolic processes. One of its most striking clinical uses is in the treatment of alcoholic liver cirrhosis that, until now, remained medically untreatable. Mato et al, in 1999, demonstrated the ability of oral S-adenosyl-L-methionine in alcoholic liver cirrhosis to decrease the overall mortality and/or progression to liver transplant by 29% vs 12% as compared with a placebo treated group. (Mato et al, (1999) S-adenosylmethionine in alcohol liver cirrhosis: a randomized, placebo-controlled, double blind, multi-center clinical trial. Journal of Hepatology, 30, 1081-1089.) The extensive clinical use of S-adenosyl-L-methionine has proven its efficacy as well as its absence of toxicity in a number of different clinical conditions. Indeed, further basic science as well as clinical studies on this very important molecule may elucidate new uses for S-adenosyl-L-methionine in medicine.

S-adenosyl-L-methionine has been used clinically in the treatment of liver disease (Friedel H, Goa, K. L., and Benfield P., (1989), S-adenosyl-L-methionine: a review of its pharmacological properties and therapeutic potential in liver dysfunction and affective disorders in relation to its physiological role in cell metabolism. Drugs. 38, 389-416), arthritis (Di Padova C, (1987), S-adenosyl-L-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosyl-L-methionine in depression: a randomized, double blind, placebo-controlled trial. Am. J. Psychiatry 147, 591-595.) Alzheimer's patients have reduced cerebral spinal fluid levels of S-adenosyl-L-methionine (Bottiglieri et al, (1990), Cerebrospinal fluid S-adenosyl-L-methionine in depression and dementia: effects of treatment with parenteral and oral S-adenosyl-L-methionine. J. Neurol. Neurosurg. Psychiatry 53, 1096-1098.)

In a preliminary study, S-adenosyl-L-methionine was able to produce cognitive improvement in patients with Alzheimer's disease. (Bottiglieri et al (1994), The clinical potential of admetionine (S-adenosyl-L-methioinine) in neurological disorders. Drugs 48, 137-152.)

S-adenosyl-L-methionine brain levels in patients with Alzheimer's disease are also severely decreased. (Morrison et al, (1996), Brain S-adenosyl-L-methionine levels are severely decreased in Alzheimer's disease, Journal of Neurochemistry, 67, 1328-1331.) S-adenosyl-L-methionine levels in patients treated with the antineoplastic drug methotrexate are reduced. Neurotoxicity associated with this drug may be attenuated by co-administration of S-adenosyl-L-methionine. (Bottiglieri et al (1994), The Clinical Potential of Ademetionine (S-adenosyl-L-methionine) in neurological disorders, Drugs, 48 (2), 137-152.)

Cerebral spinal fluid levels of S-adenosyl-L-methionine have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

Oral S-adenosyl-L-methionine administration to patients suffering from primary fibromyalgia resulted in significant improvement after a short-term trial. (Tavoni et al, Evaluation of S-adenosylmethioine in Primary Fibromaylgia. The American Journal of Medicine, Vol 83 (suppl 5A), pp 107-110, 1987.) S-adenosyl-L-methionine has been used for the treatment of osteoarthritis as well. (Koenig B. A long-term (two years) clinical trial with S-adenosyl-L-methionine for the treatment of osteoarthritis. The American Journal of Medicine, Vol 83 (suppl 5A), Nov. 20, 1987 pp 89-94)

S-adenosyl-L-methionine also attenuates the damage caused by tumor necrosis factor alpha and can also decrease the amount of tumor necrosis factor alpha secreted by cells. Consequently, conditions in which this particular inflammatory factor is elevated would benefit from the administration of S-adenosyl-L-methionine. (Watson W H, Zhao Y, Chawla R K, (1999) Biochem J Aug. 15; 342 (Pt 1):21-5. S-adenosyl-L-methionine attenuates the lipopolysaccharide-induced expression of the gene for tumour necrosis factor alpha.) S-adenosyl-L-methionine has also been studied for its ability to reduce the toxicity associated with administration of cyclosporine A, a powerful immunosuppressor. (Galan A, et al, Cyclosporine A toxicity and effect of the S-adenosyl-L-methionine, Ars Pharmaceutica, 40:3; 151-163, 1999.)

S-adenosyl-L-methionine, incubated in vitro with human erythrocytes, penetrates the cell membrane and increases ATP within the cell thus restoring the cell shape. (Friedel et al, S-adenosyl-L-methionine: A review of its pharmacological properties and therapeutic potential in liver dysfunction and affective disorders in relation to its physiological role in cell metabolism, Drugs 38 (3):389-416, 1989).

S-adenosyl-L-methionine has been studied in patients suffering from migraines and found to be of benefit. (Friedel et al, S-adenosyl-L-methionine: A review of its pharmacological properties and therapeutic potential in liver dysfunction and affective disorders in relation to its physiological role in cell metabolism, Drugs 38 (3): 389-416, 1989)

Belli et al in an article entitled “S-adenosylmethionine prevents total parenteral nutrition-induced cholestasis in the rat”, Journal of Hepatology 1994; 21: 18-23 showed that S-adenosyl-L-methionine was able to prevent cholestasis resulting from total parenteral nutrition by maintaining liver plasma membrane enzymatic activity via preservation of the membrane lipid environment.

S-adenosyl-L-methionine has been administered to patients with peripheral occlusive arterial disease and was shown to reduce blood viscosity, chiefly via its effect on erythrocyte deformability.

Garcia P et al in “S-adenosylmethionine: A drug for the brain?”, IV th Workshop on Methionine Metabolism: Molecular Mechanisms and Clinical Implications”, Symposium held on Mar. 1-5, Granada, Spain, 1998, reported that S-adenosyl-L-methionine was able to increase the number of muscarinic receptors in the brains of rats treated chronically with S-adenosyl-L-methionine. Muscarinic receptors in the brain, especially in the hippocampus, are important in learning and memory.

In a standard eight arm radical maze test, treated animals were able to out-perform age matched older untreated animals. Young aged matched S-adenosyl-L-methionine treated animals were also able to out-perform young non-treated animals showing S-adenosyl-L-methionine's ability to increase memory even in young animals. The conclusions drawn were that S-adenosyl-L-methionine is able to improve memory not only in adult aged animals but also in young animals thus making S-adenosyl-L-methionine an eligible candidate therapy for the treatment of memory impairment and learning difficulties.

Detich et al in an article entitled “The methyl donor S-adenosyl-L-methionine inhibits active demethylation of DNA; a candidate novel mechanism for the pharmacological effects of S-adenosylmethionine.” J Biol. Chem. 2003 Jun. 6; 278(23):20812-20, point out the tumor protective mechanism of S-adenosyl-L-methionine and the importance of intracellular S-adenosyl-L-methionine concentrations in cancer prevention. Presumably this is due to the ability of S-adenosyl-L-methionine to prevent or reverse global genomic DNA hypomethylation. Indeed, DNA hypomethylation is a hallmark of cancer cells and the correction of this hypomethylation leads to proper gene expression and reversal or prevention of cancer.

S-adenosyl-L-methionine is a very important molecule involved in epigenetic control of gene expression via is activity in transmethylation reactions on the genome (DNA and RNA). Thus it has been reported in the literature that S-adenosyl-L-methionine has therapeutic value in depression, arthritis, liver disease, metabolic disease, prevention or treatment of primary as well as metastatic cancer, memory impairment, aging, Alzheimer's disease and most diseases that have an epigenetic origin. Thus, it is vitally important to have a composition of S-adenosyl-L-methionine that is stable to provide the activity of methyl donor. It is only the (S,S)S-adenosyl-L-methionine diastereomer that participates in transmethylation of DNA and RNA. The (R,S)S-adenosyl-L-methionine is a methyltransferase inhibitor and thus would be completely undesirable in the event one would wish to methylate the genome for gene regulation.

The non-racemic S-adenosyl-L-methionine compositions of the present invention represent an important and needed improvement over currently existing S-adenosyl-L-methionine compositions due to the stability of the diastereomers to chiral shifting from (S,S) to the (R,S) diastereomers.

S-adenosyl-L-methionine, however, presents certain difficult problems in terms of its stability at ambient temperature that result in degradation of the molecule to undesirable degradation products as well as to the epimerization of the (S,S)S-adenosyl-L-methionine to (R,S)S-adenosyl-L-methionine. S-adenosyl-L-methionine has therefore been the subject of numerous patents directed both towards the obtaining of new stable salts, and towards the provision of preparation processes which can be implemented on an industrial scale. All citations referenced in this patent application are incorporated herein in their entirety.

Many organic compounds exist in optically active forms, i.e., they have the ability to rotate the plane of plane-polarized light. In describing an optically active compound, the prefixes D and L or R and S are used to denote the absolute configuration of the molecule about its chiral center. The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture. A compound with more than one chiral center is a diastereomer. S-adenosyl-L-methionine is a diastereomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen.

S-adenosyl-L-methionine is commercially available using fermentation technologies that result in S-adenosyl-L-methionine formulations varying between 60 and 82% purity. (That is, the final product contains 60-80% of the active or (S,S)-S-adenosyl-L-methionine and 20-40% of the inactive (in terms of transmethylation) or (R,S)-S-adenosyl-L-methionine.) (Gross, A., Geresh, S., and Whitesides, G m (1983) Appl. Biochem. Biotech. 8, 415.) Enzymatic synthetic methodologies have been reported to yield the inactive isomer (in terms of transmethylation) in concentrations exceeding 60%. (Matos, J R, Rauschel F M, Wong, C H. S-adenosyl-L-methionine: Studies on Chemical and Enzymatic Synthesis. Biotechnology and Applied Biochemistry 9, 39-52 (1987). S-adenosyl-L-methionine may also be produced using bacteria to produce the desired molecule. The highest known concentration of (S,S)-S-adenosyl-L-methionine in commercial products reported in the literature is 82.3%. Hanna, Pharmazie, 59, 2004, number 4 pp 251-256.

It has proven to be very difficult to manufacture as well as maintain such high (S,S)-S-adenosyl-L-methionine concentrations in the finished pharmaceutical products. However, the inventor has surprisingly discovered that by using the manufacturing process of the present patent application, he has been able to not only provide a very high concentration of (S,S)-S-adenosyl-L-methionine vs (R,S)-S-adenosyl-L-methionine and their pharmaceutically acceptable salts, but has also been able to maintain these relatively high concentrations, that is, within the range already discussed above.

Yeast and bacteria as a rule produce solely the (S,S)S-adenosyl-L-methionine diastereomer. However, during the extraction as well as the purification process, epimerization of the (S,S)S-adenosyl-L-methionine diastereomer to the (RS)S-adenosyl-L-methionine form takes place. An example of bacterial fermentation is disclosed in US patent application 20040175805, Leonhartsberger et al, Sep. 9, 2004.

Enantiomeric separation technologies have been reported to resolve the pure active diastereomer of S-adenosyl-L-methionine. (Matos, J R, Rauschel F M, Wong, C H. S-adenosyl-L-methionine: Studies on Chemical and Enzymatic Synthesis. Biotechnology and Applied Biochemistry 9, 39-52 (1987; Hoffman, Chromatographic Analysis of the Chiral and Covalent Instability of S-adenosyl-L-methionine, Biochemistry 1986, 25 4444-4449: Segal D and Eichler D, The Specificity of Interaction between S-adenosyl-L-methionine and a nucleolar 2-O-methyltransferase, Archives of Biochemistry and Biophysics, Vol. 275, No. 2, December, pp. 334-343, 1989) Newer separation technologies exist to resolve enantiomers and diastereomers on a large commercial production scale at a very economic cost. In addition, it would be conceivable to synthesize the biologically active diastereomer using special sterioselective methodologies but this has not been accomplished to date.

De la Haba first showed that the sulfur is chiral and that only one of the two possible configurations was synthesized and used biologically. (De la Haba et al J. Am. Chem. Soc. 81, 3975-3980, 1959) Methylation of RNA and DNA is essential for normal cellular growth. This methylation is carried out using S-adenosyl-L-methionine as the sole or major methyl donor with the reaction being carried out by a methyltransferase enzyme. Segal and Eichler showed that the enzyme bound (S,S)-S-adenosyl-L-methionine 10 fold more tightly than the biologically inactive (R,S)-S-adenosyl-L-methionine thus demonstrating a novel binding stereospecificity at the sulfur chiral center. Other methyltransferases have been reported to bind (R,S)-S-adenosyl-L-methionine to the same extent as (S,S)-S-adenosyl-L-methionine and thus (R,S)-S-adenosyl-L-methionine could act as a competitive inhibitor of that enzyme. (Segal D and Eichler D, The Specificity of Interaction between S-adenosyl-L-methionine and a nucleolar 2-O-methyltransferase, Archives of Biochemistry and Biophysics, Vol. 275, No. 2, December, pp. 334-343, 1989; Borchardt R T and Wu Y S, Potential inhibitors of S-adenosyl-L-methionine-dependent methyltransferases. Role of the Asymmetric Sulfonium Pole in the Enzymatic binding of S-adenosyl-L-methionine, Journal of Medicinal Chemistry, 1976, Vol 19, No. 9, 1099-1103.)

Borchardt and Wu, in an article entitled “Potential Inhibitors of S-adenosyl-L-methionine-dependent methyltransferases. 5. Role of the Asymmetric Sulfonium Pole in the Enzymatic Binding of Adenosyl-L-methionine”, Journal of Medicinal Chemistry, 1976, Vol. 19, No. 9, pp 1099-1103, report that the (+)-SAM (no longer used nomenclature for (R,S)-S-adenosyl-L-methionine) is a potent inhibitor of enzyme-catalyzed transmethylation reactions. Since transulferation and methylation reactions are the hallmark of S-adenosyl-L-methionine's mechanism of action, it would be prudent to use S-adenosyl-L-methionine with as enriched a concentration of (S,S)-S-adenosyl-L-methionine in any pharmaceutical composition as possible since the (R,S)-S-adenosyl-L-methionine diastereomer may be inhibitory to the desired action of (S,S)-S-adenosyl-L-methionine.

However, in light of the known inability of (R,S)-S-adenosyl-L-methionine to participate in methylation or transulfuration reactions (indeed, it inhibits these reactions), it becomes increasingly apparent that S-adenosyl-L-methionine compositions should contain the least amount of (R,S)-S-adenosyl-L-methionine possible when the activity one wishes to use clinically relates to methylation of the genome.

S-adenosyl-L-methionine (whether in its optically pure diastereomeric form or in defined non-racemic ratios of (S,S)-S-adenosyl-L-methionine to (R,S)-S-adenosyl-L-methionine or as a racemic mixture) presents certain difficult problems in terms of its stability at ambient temperature that result in degradation of the molecule to undesirable degradation products as well as to epimerization to its less desirable form (R,S)-S-adenosyl-L-methionine. S-adenosyl-L-methionine (and thus its diastereomers) must be further stabilized since they exhibit intramolecular instability that causes the destabilization and breakdown of the molecule at both high as well as ambient temperatures.

Najm et al in BMC Musculoskelet Disord. 2004 Feb. 26; 5(1):6 confronted the problem of S-adenosyl-L-methionine diastereomer instability during a double-blind cross-over trial for the treatment of osteoarthritis using S-adenosyl-L-methionine. During the course of the clinical trial, the authors noted that the S-adenosyl-L-methionine used at the beginning of the trial had epimerized to a ratio of (S,S)S-adenosyl-L-methionine/(R,S)S-adenosyl-L-methionine of 45%/55% respectively. Thus, the trial had to be halted until new batches of S-adenosyl-L-methionine could be made to continue the trial. This is a problem for all salts of S-adenosyl-L-methionine and clearly poses a quality control issue for drug development.

The present patent solves, to some extent, the quality control issues inherent in this unstable molecule. Thus, by controlling the temperature of the extraction and purification steps during the manufacturing process, the rate of epimerization of S-adenosyl-L-methionine from (S,S)S-adenosyl-L-methionine to (R,S)S-adenosyl-L-methionine can be slowed down.

All attempts at synthetic methodologies to manufacture S-adenosyl-L-methionine have resulted in racemic mixtures of 50%/50% (S,S)S-adenosyl-L-methionine to (R,S)S-adenosyl-L-methionine. Stereochemical synthesis of S-adenosyl-L-methionine has not yet been accomplished. However, the very same problem of epimerization would be raised during the manufacturing process. To overcome the epimerization of the molecule from (S,S)S-adenosyl-L-methionine to (R,S)S-adenosyl-L-methionine one would still be required to control the temperature of the synthetic reaction as well as purification (in event that the synthesis does not result in 100% (S,S)S-adenosyl-L-methionine) and the salification process. Thus the inventor anticipates that the problems of stereochemical synthesis of S-adenosyl-L-methionine will be overcome. The inventor contemplates halting of the epimerization of the S-adenosyl-L-methionine using temperature to control the rate of epimerization in the same manner as disclosed herein with regards to yeast or bacterial fermentation to obtain the S-adenosyl-L-methionine.

S-adenosyl-L-methionine has therefore been the subject of many patents directed both towards obtaining new stable salts, and towards the provision of preparation processes that can be implemented on an industrial scale. The present patent thus envisions the use of any of the salts of S-adenosyl-L-methionine already disclosed in the prior art in order to stabilize the diastereomeric forms of S-adenosyl-L-methionine disclosed in this patent. Examples of such salts disclosed in the prior art include, but not limited to, the following: a lipophilic salt of S-adenosyl-L-methionine of the formula S-adenosyl-L-methionine.sup.n+ [R—CO—NH—(CH.sub.2).sub.2—SO.sup.−.sub.3].sub.n in which R—CO is a member selected from the group consisting of C.sub.12-C.sub.26 saturated and unsaturated, linear and branched acyl and C.sub.12-C.sub.26 cycloalkyl-substituted acyl, and n is an integer from 3 to 6 according to the S-adenosyl-L-methionine charge; double salts corresponding to the formula S-adenosyl-L-methionine.sup.+.HSO.sub.4.sup.−.H.sub.2 SO.sub.4 0.2 CH.sub.3 C.sub.6H.sub.4 SO.sub0.3H.; salts (S,S)-S-adenosyl-L-methionine with sulphonic acids selected from the group consisting of methanesulphonic, ethanesulphonic, 1-n-dodecanesulphonic, 1-n-octadecanesulphonic, 2-chloroethanesulphonic, 2-bromoethanesulphonic, 2-hydroxyethanesulphonic, 3-hydroxypropanesulphonic, d-,1-,d,1-10-camphorsulphonic, d-,1-,d,1-3-bromocamphor-10-sulphonic, cysteic, benzenesulphonic, p-chlorobenzenesulphonic, 2-mesitylbenzenesulphonic, 4-biphenylsulphonic, 1-naphthalenesulphonic, 2-naphthalenesulphonic, 5-sulphosalicylic, p-acetylbenzenesulphonic, 1,2-ethanedisulphonic, methanesulphonic acid, ethanesulphonic acid, 1-n-dodecanesulphonic acid, 1-n-octadecanesulphonic acid, 2-chloroethanesulphonic acid, 2-bromoethanesulphonic acid, 2-hydroxyethanesulphonic acid, d-,1-,d,1-10-camphorsulphonic acid, d-,1-,d,1-3-bromocamphor-10-sulphonic acid, cysteic acid, benzenesulphonic acid, 3-hydroxypropanesulphonic acid, 2-mesitylbenzenesulphonic acid, p-chlorobenzenesulphonic acid, 4-biphenylsulphonic acid, 2-naphthalenesulphonic acid, 5-sulphosalicylic acid, 1,2-ethanedisulphonic acid, p-acetylbenzenesulphonic acid, 1-naphthalenesulphonic acid, o-benzenedisulphonic and chondroitinesulphuric acids, and double salts of said acids with sulphuric acid; S-adenosyl-L-methionine or a pharmaceutically acceptable salt thereof and an effective amount of a lithium salt selected from the group consisting of lithium chloride, lithium bromide, lithium iodide, lithium sulfate, lithium nitrate, lithium phosphate, lithium borate, lithium carbonate, lithium formate, lithium acetate, lithium citrate, lithium succinate and lithium benzoate; water-soluble salt of a bivalent or trivalent metal is a member selected from the group consisting of calcium chloride, ferric chloride, magnesium chloride, and magnesium sulfate; the salt of S-adenosyl-L-methionine is a member selected from the group consisting of salts of S-adenosyl-L-methionine with hydrochloric acid, sulfuric acid, phosphoric acid, formic acid, acetic acid, citric acid, tartaric acid, and maleic acid; and a double salt of S-adenosyl-L-methionine with said acids; a salt of S-adenosyl-L-methionine and a water-soluble polyanionic substance selected from the group consisting of a polyphosphate, metaphosphate, polystyrene sulfonate, polyvinyl sulfonate, polyvinyl sulfate, polyvinyl phosphate, and polyacrylate wherein the stoichiometric ratio of mols of S-adenosyl-L-methionine to gram-equivalent of the polyanionic substance is from 0.1:1 to 0.5; a salt of S-adenosyl-L-methionine wherein the polyanionic substance is a polyphosphate, para-polystyrene sulfonate or metaphosphate; a salt of the general formula: S-adenosyl-L-methionine.nR(O).sub.m (SO.sub.3H)ₚ (I) where m can be zero or 1; n is 1.5 when p is 2, and is 3 when p is 1; R is chosen from the group consisting of alkyl, phenylalkyl and carboxyalkyl, in which the linear or branched alkyl chain contains from 8 to 18 carbon atoms, and in particular for producing S-adenosyl-L-methionine salts of sulphonic acids, or of sulphuric acid esters, or of dioctylsulphosuccinic acid.

However the more preferred salts of the S-adenosyl-L-methionine diastereomers are chosen from the group consisting of salts (either single or double) of S-adenosyl-L-methionine diastereomers with sulfuric acid, p-toluenesulfonic acid, and 1,4-butanedisulfonate.

It is an object of the present invention to provide a method of extracting and purification of the S-adenosyl-L-methionine at temperatures between 1-10 degrees C. to prevent, halt or slow down the epimerization of (S,S)S-adenosyl-L-methionine to (RS)S-adenosyl-L-methionine.

It is a further object of the present invention to provide a method related to the use of compositions of the present invention with high (S,S) to (R,S)diastereomer ratios to increase blood, and other tissue and fluid levels of S-adenosyl-L-methionine and to increase DNA and RNA methylation levels in situations in which hypomethylation of DNA and RNA is an underlying factor and to treat conditions which result from low blood and tissue levels of S-adenosyl-L-methionine and of DNA and RNA hypomethylation. There is also a need in the art for synthetic routes to make such new compositions. The author of this present invention fulfills these needs and provides further related advantages.

### SUMMARY OF THE INVENTION

In one embodiment, a method is disclosed to prevent, halt or slow down the epimerization of (S,S)S-adenosyl-L-methionine to (R,S)S-adenosyl-L-methionine during the extraction and purification process by keeping the temperature at which these procedures are carried out between 1 and 10 degrees C. This is an important step since it is another embodiment of the current patent to provide compositions of stable salts of stable salts of optically pure and defined non-racemic ratios of (S,S)S-adenosyl-L-methionine to (R,S)S-adenosyl-L-methionine.

In another embodiment, a method of treatment of diseases associated with DNA or RNA hypomethylation, low blood or tissue levels of S-adenosyl-L-methionine using the non-racemic S-adenosyl-L-methionine obtained by the process method disclosed.

### DETAILED DESCRIPTION OF THE INVENTION

As mentioned above, this invention is generally directed to defined non-racemic S-adenosyl-L-methionine and its pharmaceutically acceptable salts, when administered to a warm blooded animal in need thereof, have utility in the prevention or treatment of conditions associated with low levels of S-adenosyl-L-methionine in warm blooded animals, including humans or in lowered DNA or RNA methylation levels.

S-adenosyl-L-methionine is commercially available using fermentation technologies that result in S-adenosyl-L-methionine formulations varying between 60 and 80% purity. (That is, the final product contains 60-80% of the active or (S,S)-S-adenosyl-L-methionine and 20-40% of the inactive or (R,S)-S-adenosyl-L-methionine.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-Adenosylmethionine: Studies on Chemical and Enzymatic Synthesis. Biotechnology and Applied Biochemistry 9, 39-52 (1987).

A recent US patent application 20020188116 Deshpande, Pandurang Balwant; et al. Dec. 12, 2002 entitled “Chemical synthesis of S-adenosyl-L-methionine with enrichment of (S,S)-isomer.” discloses methodology to synthesize S-adenosyl-L-methionine but does not disclose any methodology to stabilize the molecule once its synthesized. In addition, Deshpande et al do not disclose the process of controlling the temperature of the synthetic reaction. U.S. Pat. No. 6,958,233 Berna, Marco; et al. Nov. 21, 2002 entitled “Process for the preparation of pharmaceutically acceptable salts of (R,S)-S-adenosyl-L-methionine” disclose a process for the preparation of a relatively purified biologically active diastereomer (S,S)S-adenosyl-L-methionine (97%) but does not disclose the use of non-racemic S-adenosyl-L-methionine diasteromer mixtures that are the subject of this current invention.

S-adenosyl-L-methionine (whether in its optically pure diastereomeric form or in a non-racemic mixture) presents certain difficult problems in terms of its stability at ambient temperature that result in degradation of the molecule to undesirable degradation products. S-adenosyl-L-methionine (and thus its diastereomers) must be further stabilized since it exhibits intramolecular instability that causes the destabilization and breakdown of the molecule at both high as well as ambient temperatures. S-adenosyl-L-methionine has therefore been the subject of many patents directed both towards obtaining new stable salts, and towards the provision of preparation processes that can be implemented on an industrial scale.

As used herein, the term “conditions” includes diseases, injuries, disorders, indications and/or afflictions that are associated with decreased levels of S-adenosyl-L-methionine and methylation of DNA and RNA. The term “treat” or “treatment” means that the symptoms associated with one or more conditions associated with low levels of S-adenosyl-L-methionine are alleviated or reduced in severity or frequency and the term “prevent” means that subsequent occurrences of such symptoms are avoided or that the frequency between such occurrences is prolonged.

The term “modulation” as used herein means, as it is applied to the target gene, regulated, adjusted, or adapted to a desired degree that results in the desired degree of expression of the protein that the gene encodes.

As used herein, the term “gene” refers to all nucleotide sequences associated with a gene, including coding sequences; non-coding sequences such as 5′ and 3′ untranslated regions and introns, as well as any other sequences containing elements that regulate transcription of the gene, such as promoter regions. The “template” strand of a gene is used to transcribe RNA in a reverse (non-sense) direction. The sense strand of DNA is complementary to the template strand. The target DNA sequence can be on either strand of DNA.

The phrase “gene regulatory region” refers to regions including nucleotide sequences containing elements that regulate transcription of a gene, including but not limited to promoters, enhancers, splicing sites, 5′-regulatory or 3′-regulatory regions, suppressors, and silencers.

As used herein, the terms “disease” and “disorder” refer to any condition of an organism that impairs normal physiological functioning.

As used herein, “a disease gene” is any gene whose expression, underexpression or overexpression correlates with a disease or disorder.

As used herein, the term “conditions” includes diseases, injuries, disorders, indications and/or afflictions that are associated with decreased levels of S-adenosyl-L-methionine or decreased levels of genomic DNA or RNA methylation (termed hypomethylation). The term “treat” or “treatment” means that the symptoms associated with one or more conditions associated with low levels of S-adenosyl-L-methionine and decreased levels of genomic DNA or RNA methylation are alleviated or reduced in severity or frequency and the term “prevent” means that subsequent occurrences of such symptoms are avoided or that the frequency between such occurrences is prolonged.

The term “defined non-racemic” mixture or ratio of (S,S)S-adenosyl-L-methionine to (R,S)S-adenosyl-L-methionine includes compositions employed in the methods of use of the present invention wherein the defined non-racemic ratio of (S,S)S-adenosyl-L-methionine to (R,S)S-adenosyl-L-methionine is about 82.5% to 96.99%: 17.5% to 2.01% by weight respectively.

Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

In a preferred embodiment, substantially optically pure diastereomeric forms of S-adenosyl-L-methionine salts or a non-racemic mixture of (S,S)-S-adenosyl-L-methionine and (R,S)-S-adenosyl-L-methionine and their salts of this current patent application are administered to a warm-blooded animal as a pharmaceutical, prophylactic or cosmetic composition containing at least one substantially optically pure diastereomeric form of S-adenosyl-L-methionine salt or a non-racemic mixture of (S,S)-S-adenosyl-L-methionine and (R,S)-S-adenosyl-L-methionine and their salts in combination with at least one pharmaceutically, prophylactically or cosmetically acceptable carrier or diluent. Administration may be accomplished by systemic or topical application, with the preferred mode dependent upon the type and location of the conditions to be treated. Frequency of administration may vary, and is typically accomplished by daily administration. Typical oral dosages may range for humans from about 100 mg per day to about 3 grams per day and more given in divided doses throughout the day. IV and IM dose ranges from about 100 mg per day to about 3 grams per day for humans.

For prophylactic or therapeutic applications, the dose administered to an individual, in the context of the present invention, should be sufficient to effect a beneficial response in the individual over time (i.e., an effective amount). This amount, which will be apparent to the skilled artisan, depends on the species, age, and weight of the individual; the type of disease to be treated; in some cases the sex of the individual; and other factors which are routinely taken into consideration when treating individuals at risk for, or having, a disease. A beneficial effect is assessed by measuring the effect of the compound on the disease state in the individual. For example, if the disease to be treated is cancer, the therapeutic effect can be assessed by measuring the growth rate or the size of the tumor; by measuring the production of compounds, such as cytokines, that indicate progression or regression of the tumor; and by mortality. More specifically, one may wish to follow patient's progress via blood levels of, for example, urokinase, VEGF, MMP, or to assess LINE-1 hypomethylation status of lymphocytes or any other of a number of currently acceptable methods to assess effect of modulated methylation of genes and proteins in cells.

Dosing is dependent on the severity and responsiveness of the disease state to be treated or prevented, with the course of treatment lasting until a beneficial effect is achieved or, in the case of prophylaxis, for as long as required to prevent onset of the disease. Optimal dosing schedules can be calculated from measurements of drug accumulation in the body of the individual. Persons of ordinary skill can readily determine optimum dosages, dosing methodologies, and repetition rates. Persons of ordinary skill in the art can readily estimate repetition rates for dosing based on measured residence times and concentrations of the administered stable substantially optically pure or defined non-racemic S-adenosyl-L-methionine, its diastereomers and their pharmaceutically acceptable salts in bodily fluids or tissues.

Typical oral dosages for the treatment of the conditions listed above lie in the range of from 100 mg to 1600 mg or greater per day given in divided doses orally or by other routes of delivery currently used. Typical IM or IV dosages are in the range of between 200 mg and 1200 mg daily continuous or divided.

Owing to their simple conception and low costs, the procedures described in this invention easily lend themselves to working out methods of preparation on an industrial scale.

S-adenosyl-L-methionine is commercially available using fermentation technologies that result in S-adenosyl-L-methionine formulations varying between 60 and 82% purity. (That is, the final product contains 60-80% of the active or (S,S)-S-adenosyl-L-methionine and 20-40% of the inactive or (R,S)-S-adenosyl-L-methionine.) (Gross, A., Geresh, S., and Whitesides, Gm (1983) Appl. Biochem. Biotech. 8, 415.) Enzymatic synthetic methodologies have been reported to yield the inactive isomer in concentrations exceeding 60%. (Matos, J R, Rauschel F M, Wong, C H. S-Adenosylmethionine: Studies on Chemical and Enzymatic Synthesis. Biotechnology and Applied Biochemistry 9, 39-52 (1987). United States Patent Application 20020173012 Berna, Marco; et al. Nov. 21, 2002 entitled “Process for the preparation of pharmaceutically acceptable salts of (R,S)-S-adenosyl-L-methionine” disclose a process for the preparation of a relatively purified biologically active diastereomer (S,S)S-adenosyl-L-methionine (97%) but does not disclose the use of the non-racemic concentrations that are the object of this present invention.

S-adenosyl-L-methionine (whether in its optically pure diastereomeric form or in an enantiomeric or racemic mixture) presents certain difficult problems in terms of its stability at ambient temperature that result in degradation of the molecule to undesirable degradation products. S-adenosyl-L-methionine (and thus its diastereomers) must be further stabilized since it exhibits intramolecular instability that causes the destabilization and breakdown of the molecule at both high as well as ambient temperatures. In addition, the molecule, S-adenosyl-L-methionine consists of diastereomers as discussed above. The molecule is diasteromerically unstable both in solution as well as on the shelf. S-adenosyl-L-methionine has therefore been the subject of many patents directed both towards obtaining new stable salts, and towards the provision of preparation processes that can be implemented on an industrial scale.

While the problem related to the intramolecular instability of S-adenosyl-L-methionine has been relatively successfully addressed using a variety of methods ranging from high molecular weight counter ions to polyaminonic as well as polycationic polymers, the problem of chiral instability and epimerization from (S,S)S-adenosyl-L-methionine to (R,S)S-adenosyl-L-methionine remains. However, S-adenosyl-L-methionine salts, as active pharmaceutical ingredients, must be further handled in order to make pills, capsules, lotions, injections and the like. Because of the very unstable nature of the S-adenosyl-L-methionine diastereomers (that is, the ready conversion of the (S,S)diastereomer to its undesirable (R,S) configuration) no currently available commercial S-adenosyl-l-methionine products contain an (S,S)S-adenosyl-l-methionine vs (R,S)S-adenosyl-l-methionine concentration greater than 82.4% vs 17.6% by weight respectively.

Although little data is presented in the scientific literature dealing with the chiral stability of the S-adenosyl-L-methionine commercially available, as mentioned above, it is known that they are not very stable. Berna et al in U.S. Pat. No. 6,958,233 disclose the diastereomeric instability of S-adenosyl-L-methionine 1,4 butanedisulfonate available commercially and known as Samyr. The (S,S)S-adenosyl-L-methionine vs (R,S)S-adenosyl-L-methionine in vials was 58% vs 42% by weight respectively. Berna et al also evaluated the diastereomeric ratios in Smyr tablets and found the following ratios: (S,S)S-adenosyl-L-methionine vs (R,S)S-adenosyl-L-methionine in tablets was 59% vs 41% by weight respectively. Thus, the chiral instability of currently available S-adenosyl-l-methionine tablets and powder for IV or IM administration is now known. There exists a need in the art for more chirally stable S-adenosyl-L-methionine and pharmaceutically acceptable salts.

It is one object of the present invention to provide a composition useful for the treatment of conditions in which lowered levels of methylation in genomic DNA or RNA, cell, tissue or blood play a role in pathology, comprising an effective amount of S-adenosyl-L-methionine produced by yeast fermentation, extracted and purified by methods known in the art in the temperature range of between 2 and 10 degrees centigrade to maintain defined non-racemic ratio of (S,S)S-adenosyl-L-methionine to (R,S)S-adenosyl-L-methionine between 82.5%-96.99%/15.5%-2.01% by weight respectively, and salified using a pharmaceutically acceptable acid to stabilize the resulting defined non-racemic ratio of (S,S)S-adenosyl-L-methionine to (R,S)S-adenosyl-L-methionine and then drying the resulting solution to obtain a powder stable for at least one year.

Pathological conditions associated with lowered blood or tissue levels of S-adenosyl-L-methionine or of DNA or RNA hypomethylation in warm-blooded mammals including humans are selected from the group consisting of depression, osteoarthritis, autoimmune disease, cardiovascular disease, primary cancer and metastatic cancers, aging, mild cognitive impairment, liver disease including non-alcoholic steatohepatitis, viral and alcohol related liver disease, Alzheimer's disease, wet and dry macular degeneration, exposure to environment chemicals that lower DNA or RNA methylation (such chemicals as arsenic, bisphenol A and others).

Thus, it is another object of the present invention to provide a method of use as well as a process for the manufacture of a non-racemic concentration of (S,S)S-adenosyl-l-methionine vs. (R,S)S-adenosyl-l-methionine in the range between from 82.5% to 96.99%: 17.5% to 2.01% (S,S)S-adenosyl-l-methionine vs (R,S)S-adenosyl-l-methionine by weight respectively.

It is yet another object of the present invention to provide a method to treat conditions associated with lowered blood or tissue levels of S-adenosyl-L-methionine or conditions associated with DNA or RNA hypomethylation selected from the group consisting of depression, osteoarthritis, autoimmune disease, cardiovascular disease, primary cancer and metastatic cancers, aging, mild cognitive impairment, liver disease including non-alcoholic steatohepatitis, viral and alcohol related liver disease, Alzheimer's disease, wet and dry macular degeneration, exposure to environment chemicals that lower DNA or RNA methylation in warm-blooded mammals including humans comprising administering to a warm-blooded animal including humans in need thereof an effective amount of the composition of non-racemic concentration of (S,S)S-adenosyl-l-methionine vs (R,S)S-adenosyl-l-methionine in the range between from 82.5% to 96.99%: 17.5% to 2.01% (S,S)S-adenosyl-l-methionine vs (R,S)S-adenosyl-l-methionine by weight respectively along with its pharmaceutically acceptable salt.

It is another object of this present invention to provide a method of modulating gene and protein expression using stable substantially optically pure or defined non-racemic S-adenosyl-L-methionine, its diastereomers and their pharmaceutically acceptable salts that specifically target hypomethylated promoter regions of genes that are abnormally turned on during cancer as well as other states, for example, aging, cardiovascular disease, autoimmune disease and the like. It is also one object of this present invention to provide a method of modulating gene expression using stable substantially optically pure or defined non-racemic S-adenosyl-L-methionine, its diastereomers and their pharmaceutically acceptable salts that may target other areas of the DNA in order to modulate genes whose expression is desirable.

It is a further object of the present invention to provide a method to modulate protein expression by introducing into a cell stable substantially optically pure or defined non-racemic S-adenosyl-L-methionine, its diastereomers and their pharmaceutically acceptable salts whose role is to methylate those proteins that need methylation to modulate their expression.

The present invention is a method to modulate gene transcription by inducing methylation in the promoter region of the gene to be modulated. DNA methylation is a powerful, endogenous molecular mechanism by which cells modulate both endogenous and exogenous genes.

Another object of this invention is to modulate genes by administration of stable substantially optically pure or defined non-racemic S-adenosyl-L-methionine, its diastereomers and their pharmaceutically acceptable salts to the hypomethylated promoter region of the gene (or other regions of the gene that are hypomethylated) to be modulated.

Typical oral dosages for the treatment of the conditions listed above lie in the range of from 100 mg to 1600 mg or greater per day given in divided doses orally. It is well known in the literature how one may arrive at the optimum dose of S-adenosyl-L-methionine to treat or prevent a particular condition. It is well within the art to determine such doses that in any event will vary from patient population as well as clinical condition to be treated. The dose range discussed above is typical as noted in the literature.

Owing to their simple conception and low costs, the procedures described in this invention easily lend themselves to working out methods of preparation on an industrial scale.

The following examples illustrate the preparation process by which the non-racemic concentration of (S,S)S-adenosyl-L-methionine to (R,S)S-adenosyl-L-methionine may be made. The S-adenosyl-L-methionine used in the following examples may be obtained by any method known in the art, but the preferred method is the one that yields the highest concentration of (S,S)S-adenosyl-L-methionine to (R,S)S-adenosyl-L-methionine irrespective of the methodology. Thus, the preferred method would be one in which the temperature of the extraction, purification and salification processes would be controlled between 2 and 10 degrees C. and the resulting solution would be dried. These examples are given to illustrate the present invention, but not by way of limitation. Accordingly, the scope of this invention should be determined not by the embodiments illustrated, but rather by the appended claims and their legal equivalents.

The effect of the stable non-racemic S-adenosyl-L-methionine compositions of this invention in major depressive disorders is studied on patients aged from 18 to 65 years old. The patients receive, for example, a defined non-racemic S-adenosyl-L-methionine 1,4 butanedisulfonate (in this example, (S,S) vs (R,S) is 87% vs 13% by weight respectively) orally (1600 mg/day) for a period of about six weeks.

The improvement in the depressive syndromes is measured by means of a significant decrease in the scores on the Hamilton depression rating scale (HAM-D) as well as by the impressions received by the clinician and the patient's overall impressions. The Hamilton depression rating scale is defined by M. Hamilton in J. Neurol. Neurosurg. Psychiat., 1960, 23, 56-62.

The effect of the stable non-racemic S-adenosyl-L-methionine compositions of this invention on the pain associated with osteoarthritis is studied on patients aged from 65 to 85 years old. The patients receive, for example, a defined non-racemic S-adenosyl-L-methionine 1,4 butanedisulfonate (in this example, (S,S) vs (R,S) is 87% vs 13% by weight respectively) orally (1600 mg/day) for a period of about six weeks.

The improvement in the pain level associated with osteoarthritis is measured by means of a significant decrease in pain according to the visual analog scale as well as by the impressions received by the clinician and the patient's overall impressions. The use of the visual analog scale for measuring pain is described by Scott J, Huskisson E C. Graphic representation of pain. Pain. 1976; 2:175-184.

The effect of the stable non-racemic S-adenosyl-L-methionine compositions of this invention on liver function is studied on patients aged from 30 to 65 years old suffering from liver disease, such as for example, viral or alcoholic hepatitis, non alcohol steatohepatitis. The patients receive, for example, a defined non-racemic S-adenosyl-L-methionine 1,4 butanedisulfonate (in this example, (S,S) vs (R,S) is 87% vs 13% by weight respectively) orally (1600 mg/day) for a period of about 16 weeks.

The improvement in liver function in patients suffering from, for example, non alcohol steatohepatitis is measured using liver enzyme levels of alanine aminotransferase level, gamma-glutamyltransferase, as well as HbA1c levels. (Ref Gastroenterology. 2008 Jun. 25. Randomized, Placebo-Controlled Trial of Pioglitazone in Nondiabetic Subjects With Nonalcoholic Steatohepatitis. Aithal G P, Thomas J A, Kaye P V, Lawson A, Ryder S D, Spendlove I, Austin A S, Freeman J G, Morgan L, Webber J.)

One more embodiment of the present invention consists in a method of modulating a gene or a protein in a cell (human, animal, plant, parasite, viral, bacterial) comprising introducing into the cell a stable substantially optically pure or defined non-racemic S-adenosyl-L-methionine, its diastereomers and their pharmaceutically acceptable salts in a quantity sufficient to remethylate the promoter region of genes needing to be modulated or proteins needing to be modulated by methylation. It can be appreciated, for example, that the human genome may consist of over 30,000 genes many of which have not yet been identified. This present invention envisions modulation of any of such genes by the methods described. That is, all genes will have regulatory regions that may be modulated by methylation and, though not identified at this time, such genes and such regions may be modulated by the methods disclosed in the present invention.

In a preferred embodiment of the present invention a stable substantially optically pure or defined non-racemic S-adenosyl-L-methionine, its diastereomers and their pharmaceutically acceptable salts is introduced into the cell and results in a quantifiably increased methylation of the promoter region of the gene to be modulated or within CpG islands of the gene to be modulated or a protein to be modulated. (This of course may be accomplished in mammals including humans by oral, (or IV, IM, transmembrane or any other method known in the art) administration of the stable defined non-racemic S-adenosyl-L-methionine and the pharmaceutically acceptable salts of the present invention.)

The target gene is unmethylated in the promoter region and the gene is, or can be, expressed in the cells to be treated. The DNA methyltransferase enzyme methylates the cellular DNA in the hypomethylated region of the target gene. This methylation imprint in the region of the promoter modulates the specific gene, and the effect may be permanent because the methylation is subsequently inherited. The cells can be primary human or other mammalian cells, or permanent lines of human, animal or even plant origin.

To detect whether the method of this current invention results in the remethylation of the hypomethylated promoter region of the target gene, one can use any number of currently available techniques known to those skilled in the art to evaluate the methylation status of the promoter region of the target gene.

To name only one method that may be used to determine the methylation status of the promoter region of the target gene: the methodology presented in Cancer Res 2006; 66: (18). Sep. 15, 2006 pp 9202 9210 entitled: Alteration of the Methylation Status of Tumor-Promoting Genes Decreases Prostate Cancer Cell Invasiveness and Tumorigenesis In vitro and In vivo by Nicholas Shukeir, Pouya Pakneshan, Gaoping Chen, Moshe Szyf, and Shafaat A. Rabbani. Methodologies exist to check the methylation status of proteins and are well known to those skilled in the art. As an example, see Journal of Alzheimer's Disease 9 (2006) 415-419 415 entitled: The effect of S-adenosylmethionine on CNS gene expression studied by cDNA microarrayanalysis by Rosaria A. Cavallaro, Andrea Fuso, Fabrizio D'Anselmi, Laura Seminara and Sigfrido Scarpa.

To determine the effect of a defined non-racemic S-adenosyl-L-methionine 1,4 butanedisulfonate (in this example, 200 uM of (S,S) vs (R,S) is 87% vs 13% by weight respectively) to methylate the promoter region of a gene the following experiment is carried out:

MDA-231 cells are treated with increasing concentrations of stable substantially optically pure or defined non-racemic S-adenosyl-L-methionine, in this particular case, a defined non-racemic S-adenosyl-L-methionine 1,4 butanedisulfonate (in this example, starting with 200 uM but increasing to 500 uM (S,S) vs (R,S) is 87% vs 13% by weight respectively) every day for 6 days. New solutions of a defined non-racemic S-adenosyl-L-methionine 1,4 butanedisulfonate (in this example, (S,S) vs (R,S) is 87% vs 13% by weight respectively) are prepared daily and administered daily to the cells. At the end of the treatment period, DNA is extracted from the cells and submitted to PCR and pyrosequencing to analyze the methylation status of the promoter region of the urokinase gene. The a defined non-racemic S-adenosyl-L-methionine 1,4 butanedisulfonate (in this example, (S,S) vs (R,S) is 87% vs 13% by weight respectively) is shown to quantitatively increase the amount of methylation in the promoter region of the urokinase gene thus showing proof of principal for this method.

MDA-231 cells are treated with increasing concentrations of a defined non-racemic S-adenosyl-L-methionine 1,4 butanedisulfonate (in this example, starting with 200 uM (S,S) vs (R,S) is 87% vs 13% by weight respectively) every day for 6 days. New solutions of a defined non-racemic S-adenosyl-L-methionine 1,4 butanedisulfonate (in this example, 200 uM (S,S) vs (R,S) is 87% vs 13% by weight respectively) is prepared daily and administered daily to the cells. At the end of the treatment period, DNA is extracted from the cells and submitted to pyrosequencing to analyze the methylation status of the promoter region of the MMP gene. The defined non-racemic S-adenosyl-L-methionine 1,4 butanedisulfonate (in this example, (S,S) vs (R,S) is 87% vs 13% by weight respectively) is shown to quantitatively increase the amount of methylation in the promoter region of the MMP gene thus showing proof of principal for this method.

MDA-231 cells are treated with increasing concentrations of a defined non-racemic S-adenosyl-L-methionine 1,4 butanedisulfonate (in this example, starting with 200 uM (S,S) vs (R,S) is 87% vs 13% by weight respectively) every day for 6 days. New solutions of the defined non-racemic S-adenosyl-L-methionine 1,4 butanedisulfonate (in this example, 200 uM (S,S) vs (R,S) is 87% vs 13% by weight respectively) are prepared daily and administered daily to the cells. At the end of the treatment period, DNA is extracted from the cells and submitted to pyrosequencing to analyze the methylation status of the promoter region of the VEGF gene. The defined non-racemic S-adenosyl-L-methionine 1,4 butanedisulfonate (in this example, (S,S) vs (R,S) is 87% vs 13% by weight respectively) is shown to quantitatively increase the amount of methylation in the promoter region of the VEGF gene thus showing proof of principal for this method of gene modulation.

The above methods show proof of principle for gene expression modulation using the compositions of the present invention.

### Example 1

S-adenosyl-L-methionine purification and extraction procedures from yeast are carried out at a temperature of between 2-10 degrees. C. These procedures for the extraction and purification are well known in the industry and have been disclosed in the prior art section and are incorporated herein in their entirety by reference. See 3,893,999, Fiecchi et al for discussion on extraction and purification. Any technique may be used to break the yeast cells to liberate the S-adenosyl-L-methionine but the preferred method is that which is carried out at temperatures between 2 and 10 degrees Centigrade. Yeast cell breakage may be carried out by mechanical means.

Salification of S-adenosyl-L-methionine using 1,4 butanedisulfonic is carried out according to Gennari U.S. Pat. No. 4,465,672 with the exception that the temperature of the procedures is within 2 degrees C. and 10 degrees C. Any pharmaceutically acceptable salt known in the literature to stabilize the molecule may be used. For example, the procedures of Fiecchi or of Gennari (U.S. Pat. No. 4,465,672) carried out at the temperatures disclosed in the present patent will result in an (S,S)S-adenosyl-L-methionine/(R,S)S-adenosyl-L-methionine concentration of between 90%-96.99% (S,S)S-adenosyl-L-methionine vs 10%-3.01% (R,S)S-adenosyl-L-methionine by weight. After drying, the aforementioned concentration of S-adenosyl-L-methionine diastereomers will remain within the stated range of the claims for 5 months. See Hanna for procedures to determine diastereomeric concentrations using NMR.

The steps for extraction and purification are outlined below:

They are prepared by a process comprising the following stages:

(a) preparing a concentrated aqueous solution of a crude SAM salt by any known method;

(b) purifying the solution by chromatography, by passage through a weakly acid ion exchange resin column;

(c) eluting the SAM with a dilute aqueous solution of the required acid;

(d) titrating the eluate and adjusting the acid quantity to the strictly stoichiometric proportion relative to the SAM present;

(e) concentrating the eluate;

(f) lyophilization or other method of drying.

The aqueous solution prepared in stage (a) can obviously contain any soluble SAM salt because the anion is eliminated in the next passage through the column, and therefore does not interfere with the rest of the process.

In all cases, the pH of the solution is adjusted to between 6 and 7, and preferably 6.5.

The chromatographic purification stage (b) is carried out preferably with Amberlite IRC50 or Amberlite CG50.

The elution of stage (c) is preferably carried out with a 0.1 N aqueous solution of the required acid. If titration of the eluate (stage d) shows that the quantity of acid equivalents present is less than 5, this being the usual case, then that quantity of acid corresponding exactly to the deficiency is added in the form of a concentrated commercial aqueous solution. However, if it is shown that an excess of acid is present, this is eliminated by treating the solution with strong basic ion exchange resin in OH.sup.− form, for example Amberlite IRA-401.

In stage (e), the eluate is concentrated to an optimum value for the subsequent lyophilization process, i.e. to a value of between 50 and 100 g/l, and preferably around 70 g/l.

The final drying is carried out by the usual methods, to give a perfectly crystalline salt of 100% purity.

The composition was stored at room temperature for two years in closed containers and the diastereomeric stability as reported in percentage of (S,S)diastereomer was assessed according to the following protocol:

Isocratic high performance liquid chromatographic analysis of S-adenosylmethionine and S-adenosylhomocysteine in animal tissues: the effect of exposure to nitrous oxide. Bottiglieri, T. (1990) Biomed Chromatogr, 4(6):239-41. An example of the methodology to determine the percentage of diastereomers of S-adenosyl-L-methionine is also well known and a new NMR technique has recently been published. Hanna, Pharmazie, 59, 2004, number 4 pp 251-256.

Diastereomeric stability data of non-racemic S-adenosyl-L-methionine 1,4 butanedisulfonate of the present invention. The data represents an average of 5 lots of samples analyzed immediately after synthesis (initial) and an average of these same 5 lots of samples analyzed two years later (final).

| | Initial (S,S) % | Final (S,S) % |
|---|---|---|
| (S,S) S-adenosyl-L-methionine as 1, | 96.44% | 86.624% |
| 4 butanedisulfonate salt | | |

I claim:

1. A method to treat conditions associated with lowered blood or tissue levels of S-adenosyl-L-methionine or conditions associated with DNA or RNA hypomethylation selected from the group consisting of depression, osteoarthritis, autoimmune disease, cardiovascular disease, primary cancer and metastatic cancers, aging, mild cognitive impairment, liver disease including non-alcoholic steatohepatitis, viral and alcohol related liver disease, Alzheimer's disease, wet and dry macular degeneration, exposure to environment chemicals that lower DNA or RNA methylation in warm-blooded mammals, including humans, wherein the method comprises administering to a warm-blooded animal, including humans in need thereof, an effective amount of the composition comprising an effective amount of S-adenosyl-L-methionine produced by yeast fermentation, extracted and purified by methods known in the art in the temperature range of between 2 and 10 degrees centigrade to maintain defined non-racemic ratio of (S,S)S-adenosyl-L-methionine to (R,S)S-adenosyl-L-methionine between 82.5%-96.99%/15.5%-2.01% by weight respectively, and salified using a pharmaceutically acceptable acid to stabilize the resulting defined non-racemic ratio of (S,S)S-adenosyl-L-methionine to (R,S)S-adenosyl-L-methionine and then drying the resulting solution to obtain a stable powder.

2. A process of preparing a powder form of a pharmaceutically acceptable salt of non-racemic S-adenosyl-L-methionine comprising the (S,S)diastereomer of the pharmaceutically acceptable salt of S-adenosyl-L-methionine in a concentration range of between 82.5% and 96.99% by weight for a period of time from 5 months to 2 years, wherein the process comprises the following steps:
a) producing S-adenosyl-L-methionine by yeast fermentation under suitable conditions; extracting and purifying the S-adenosyl-L-methionine at a temperature of between 2° C. and 10° C. under suitable conditions to produce an aqueous composition having a concentration of the (S,S)diastereomer of S-adenosyl-L-methionine of greater than 90% by weight;
b) salifying the aqueous composition with a pharmaceutically acceptable acid at a temperature of between 2° C. and 10° C. under suitable conditions;
c) concentrating the resulting aqueous composition under suitable conditions; and
d) drying the concentrate under suitable conditions to produce the powder form of the pharmaceutically acceptable salt of non-racemic S-adenosyl-L-methionine comprising the (S,S)diastereomer of the pharmaceutically acceptable salt of S-adenosyl-L-methionine in a concentration range of between 82.5% and 96.99% by weight for a period of time from 5 months to 2 years.

3. The process of claim 2 wherein the pharmaceutically acceptable acid in step b) is 1,4-butanedisulphonic acid and the pharmaceutically acceptable salt is 1,4-butane disulphonate.

4. The process of claim 2 or 3 wherein the process of drying in step d) is by lyophilization.
